(19) United States
(12) Patent Application Publication
(10) Pub. No.: US 2007/0098768 A1
(43) Pub. Date: May 3, 2007

(54) Two-sided personal-care appliance for health, hygiene, and/or environmental application(s); and method of making said two-sided personal-care appliance

(76) Inventors: Kenneth B. Close, New London, WI (US); Jonathan K. Arendt, Appleton, WI (US); Gary V. Anderson, Larsen, WI (US); Wael R. Joseph, Appleton, WI (US); Bhaskara C. Chilakamarri, Appleton, WI (US)

Correspondence Address: KIMBERLY-CLARK WORLDWIDE, INC., 401 NORTH LAKE STREET, NEENAH, WI, 54956, US

(21) Appl. No.: 11/265,391

(22) Filed: Nov. 1, 2005

(51) Int. Cl. A61K 9/70; B32B 27/04

(52) U.S. Cl. 424/443; 442/123

(57) ABSTRACT

Disclosed is a two-side personal-care appliance for various applications, including exfoliation of skin. The appliance comprises a first interbonded fibrous layer having three-dimensional contours on both faces of the layer. These contours facilitate exfoliation and/or stimulation and/or gentle abrasion of skin. Furthermore, these contours facilitate contact and attachment to the second interbonded fibrous layer. The second interbonded fibrous layer comprises a high-loft material capable of gently cleaning skin, holding liquid, and generating lather.

People use various personal-care appliances for a number of health, hygiene, and/or environmental applications.

Personal-care appliances, such as synthetic scrubbing pads or pumice stones, may be used to clean, stimulate, and/or exfoliate skin. The human body forms areas of thick, hardened, dead skin in response to the effects of pressure, friction, or injury to such areas of skin, in order to protect the skin and body structures under the skin. These areas of dead skin typically form on hands and feet, and are commonly known as calluses or corns., Calluses and corns can become problematic when they grow large enough to cause pain.

One element of accepted medical treatment at home for painful calluses and corns is to soak the affected area in warm water and then use a device such as a pumice stone to carefully abrade the dead skin away. But use of pumice stones can pose problems. Pumice stones are composed of spherically shaped glass bubbles that break. This breakage produces sharp scooping surfaces with associated cavities. These cavities tend to hold exfoliated skin after the stone used. It is difficult to completely remove the skin held in these cavities, and various kinds of microbiological growths may result. Furthermore, pumice stones, while effective in abrading the surfaces of corns and calluses, can be unduly abrasive to the finger tips of some users. Also, users, when traveling, may not want to take a pumice stone with them.

Therefore, what is needed is a personal-care appliance that can be used to abrade moist skin that is: 1) low enough in cost so as to be capable of being adapted for limited use (e.g., disposable after 1 or 2 uses), thus convenient for use away from home and overcoming the need to cleanse the device after use in an effort to prevent or reduce microbiological growth, 2) maintains integrity when wet, 3) has a surface that is gentle enough against the surface of finger tips so as to be comfortable, yet has another surface that is abrasive enough to abrade the surfaces of calluses and corns; and (4) is capable, if desired, of employing a cleaning and/or moisturizing composition or formulation. Furthermore, processes for making said personal-care appliances are needed.

### SUMMARY

We have found that a two-sided personal-care appliance comprising a first interbonded fibrous layer having three-dimensional contours (which are optionally contiguous with shaped discontinuities in said interbonded fibrous layer) that is interlocked with a second interbonded fibrous layer comprising a high-loft material is capable of balancing seemingly contradictory properties desired by users of such appliances, and, when needed, of being adapted for limited use by users of such appliances. We have found that such two-sided personal-care appliances may be used for: applying and/or utilizing cleaning compositions or other formulations (whether contained in the appliance or applied separately), and that typically the second interbonded fibrous layer comprising a high-loft material will be used for this purpose; and exfoliating, stimulating, and/or gently abrading the skin or tissue of a user, with the first interbonded fibrous layer having three-dimensional contours being used generally for this purpose.

FIG. 1 representatively depicts one version of a personal-care appliance 1 of the present invention. The personal-care appliance comprises a first interbonded fibrous layer 3 comprising three-dimensional contours and a second interbonded fibrous layer 5 comprising a high-loft material and interlocked to said first interbonded fibrous layer. In this version of a personal-care appliance of the present invention, the three dimensional contours of the first interbonded fibrous layer extend both away from the personal-care appliance, with the contours and fibers adapted for stimulating and/or exfoliating the skin of a user of the personal care appliance (i.e., the three-dimensional contours are associated with the face or surface oriented outward from first interbonded fibrous layer, and therefore outward from the personal-care appliance); and into the second interbonded fibrous layer, thereby hooking and/or otherwise interlocking with portions of said second layer (i.e., the three-dimensional contours are associated with the opposing surface or face oriented inward from the first interbonded layer and toward a surface or face of the second interbonded fibrous layer to which the first interbonded layer is attached). While not depicted in FIG. 1, it should be noted that an additional element, reinforcing strands, may be formed and attached to at least a portion of the first interbonded fibrous layer. Reinforcing strands are discussed in more detail in the Description section below.

FIGS. 1A and 1B depict representative images of a personal-care appliance of the present invention (the numerals in these Figures signify the same elements as are signified in FIG. 1 above). In FIG. 1A, numeral 5 signifies the side of the high-loft, second interbonded fibrous layer of the personal-care appliance. Numeral 3 in FIG. 1A signifies the top of the first interbonded fibrous layer having three-dimensional contours. In FIG. 1B, numeral 5 signifies the side of the high-loft, second interbonded fibrous layer of the personal-care appliance. Numeral 3 in FIG. 1B signifies the side of the first interbonded fibrous layer having three-dimensional contours. In both of these images, an adhesive (not shown) was used to attach a surface of the first interbonded fibrous layer to a surface of the second interbonded fibrous layer.

In this application, “three-dimensional contours” or a “three-dimensional topography” signifies a topography readily discernible by the human eye (e.g., changes in elevation of about 0.1 millimeter or more—suitably of about 0.5 millimeter or more—from the base of a “valley” to the top of a neighboring “ridge” in the surface of the interbonded fibrous layer; a “valley” signifies a low point or depression in the first interbonded fibrous layer that is closer to the surface of the second interbonded fibrous layer to which the first layer is attached; a “ridge” signifies a high point or elevation in the first interbonded fibrous layer that is farther away from the surface of the second interbonded fibrous layer to which the first layer is attached). Such topographies are contrasted with the topography associated with a flat sheet of writing paper, or a flat, unembossed sheet of toilet tissue. Such substrates, under a microscope, reveal surfaces having a microscopic three-dimensional topography. But such topographies are to be distinguished from the three-dimensional topographies discussed herein with respect to surfaces of interbonded fibrous layers.

We have found that the aforementioned personal-care appliances provide: (1) via the first interbonded fibrous layer having three-dimensional contours, a component and/or surface capable of stimulating and/or exfoliating and/or gently abrading the skin of a user of the personal-care appliance; and (2) via the second interbonded fibrous layer, which is formed of a high-loft, compressible material, a layer and/or surface capable of holding liquid, forming bubbles (e.g., through “pumping” or squeezing the layer during use), and cleaning the skin of a user of the appliance. Furthermore, valleys or depressions in the first interbonded fibrous layer can serve to hold dead skin that is abraded from a user's foot, hand, elbow, or other body location. Also, the second interbonded fibrous layer can be softer, pliable, and easier and/or more comfortable to hold. Finally, in the depicted embodiment, the three-dimensional contours of the first interbonded fibrous layer extend into the second interbonded layer, thereby hooking and/or interlocking with said second interbonded fibrous layer.

During development of the present invention, we found that rubber belts normally used as conveyor belts could be used to form the first interbonded fibrous layer having three-dimensional contours. Unlike conventional forming wires, such belts are readily processed to form openings in the belt (e.g., by die-cutting, drilling, puncturing, or otherwise creating openings in the belt, including by molding the belt to have openings). When the first interbonded fibrous layer is being formed on this support (e.g., a conveyor belt comprising openings), the fibrous layer over or near the openings can be pulled into (in this case by a vacuum—but positive pressure or other mechanical or other methods for applying a force to the fibrous layer may be used) the openings to provide three-dimensional contours in the fibrous layer. I.e., at least a portion of the three-dimensional contours in the first interbonded fibrous layer corresponded to, and were formed in, the openings in the supporting belt. These conveyor belts and their analogues are available with various textured surfaces. By selecting conveyor belts with different textures, additional three-dimensional contours were introduced to the first interbonded fibrous layer by the textured surface of the belt. Thus use of conveyor belts not only provide the ability to readily incorporate openings or depressions/valleys of various sizes, shapes, and placement in the belt (including, for example, the cutting of recognizable shapes such as flowers, animals, a company's logo or trademark, or other such symbol or image) such that the corresponding shaped discontinuity in the interbonded fibrous layer takes on such recognizable shape in the belt), but the ability to impart a textured surface to the interbonded fibrous layer that corresponds to the textured surface of the belt.

These and other versions, embodiments, and examples of the invention are discussed elsewhere in this application.

### DRAWINGS

FIG. 1 depicts a representative version of a personal-care appliance of the present invention. FIG. 1A depicts an image of a representative version of a personal-care appliance of the present invention. FIG. 1B depicts an image of a representative version of a personal-care appliance of the present invention.

FIG. 2 depicts a representative version of a process for making the first interbonded fibrous layer of the present invention (including any optional reinforcing strands).

FIG. 3 depicts a representative version of a process for making the second interbonded fibrous layer of the present invention.

FIGS. 4 through 7 illustrate in greater detail representative versions of forming surfaces having different textures and/or topographies. FIGS. 4A, 5A, 6A, and 7A show cross-sections taken along lines 4A-4A, 5A-5A, 6A-6A, and 7A-7A in the respective figures.

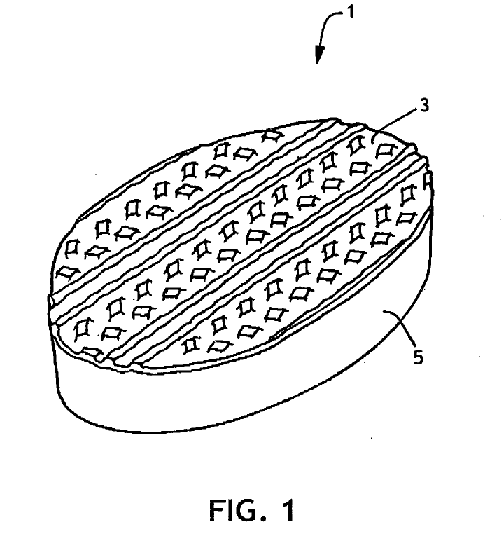

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Autogenous bonding” and its derivatives refer to bonding provided by fusion and/or self-adhesion of fibers and/or filaments without an applied external adhesive or bonding agent. Autogenous bonding may be provided by contact between fibers and/or filaments while at least a portion of the fibers and/or filaments are semi-molten or tacky. Autogenous bonding may also be provided by blending a tackifying resin with the thermoplastic polymers used to form the fibers and/or filaments. Fibers and/or filaments formed from such a blend can be adapted to self-bond with or without the application of pressure and/or heat. Solvents may also be used to cause fusion of fibers and filaments which remains after the solvent is removed.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding. “Autogenous bonding,” as described above, is a type of “bonding.” “Interbonded” and its derivatives is a type of “bonding.”

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Cleaning composition”, “cleaning formulation,” or their derivatives refer to personal care or cleaning formulations or compositions, shampoos, lotions, body washes, hand sanitizers, bar soaps, etc., whether in the form of a solid, liquid, gel, paste, foam, or the like. “Cleaning compositions” also encompass moisturizing formulations.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and-its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Hydrophilic” describes fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Layer” when used in the singular can have the dual meaning of a single element or a plurality of elements.

“Liquid impermeable,” when used in describing a layer or multi-layer laminate means that liquid will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface or support-to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes. The basis weight of nonwoven webs or materials is usually expressed in ounces of material per square yard (osy) or grams per square meter (gsm), and the fiber diameters are usually expressed in microns. (Note: to convert from osy to gsm, multiply osy by 33.91.)

“Z-direction” refers to fibers disposed outside of the plane of orientation of a web. A web will be considered to have an x-axis in the machine direction, a y-axis in the cross machine direction and a z-axis in the loft direction, with its major planes, or surfaces, lying parallel with the x-y plane. The term “as formed z-direction fibers” may be used herein to refer to fibers that become oriented in the z-direction during forming of the nonwoven web as distinguished from fibers having a z-direction component resulting from post-forming processing of the nonwoven web, such as in the case of mechanically crimped or creped or otherwise disrupted nonwoven webs.

“Substantially continuous fibers” refers to fibers which are not cut from their original length prior to being formed into a nonwoven web or fabric. Substantially continuous fibers may have average lengths ranging from greater than about 15 centimeters to more than one meter, and up to the length of the web or fabric being formed. The definition of “substantially continuous fibers” includes fibers which are not cut prior to being formed into a nonwoven web or fabric, but which are later cut when. the nonwoven web or fabric is cut, and fibers which are substantially linear or crimped.

“Through-air bonding” or “TAB” means the process of bonding a nonwoven, for example a bicomponent fiber web, in which air which is sufficiently hot to melt one of the polymers of which the fibers of the web are made is forced through the web.

“Side by side fibers” belong to the class of bicomponent or conjugate fibers. The term “bicomponent fibers” refers to fibers which have been formed from at least two polymers extruded from separate extruders but spun together to form one.fiber. Bicomponent fibers are also sometimes referred to as conjugate fibers or multicomponent fibers. Bicomponent fibers are taught, e.g., by U.S. Pat. No. 5,382,400 to Pike et al. The polymers of conjugate fibers are usually different from each other though some conjugate fibers may be monocomponent fibers. Conjugate fibers are taught in U.S. Pat. No. 5,108,820 to Kaneko et al., U.S. Pat. No. 4,795,668 to Krueger et al. and U.S. Pat. No. 5,336,552 to Strack et al. Conjugate fibers maybe used to produce crimp in the fibers by using the differential rates of expansion and contraction of the two (or more) polymers.

“Low machine direction orientation” and “high machine direction orientation” as used herein refers to the degree to which the fibers of a nonwoven web are allowed to disperse over the cross direction of the forming surface, e.g. a belt or other support; or a foraminous wire. Low machine direction orientation fibers are dispersed across the cross direction to a higher degree than a collection of fibers exhibiting a higher machine direction orientation which have less dispersion over the cross direction of the forming surface during the formation of a web.

Words of degree, such as “about”, “substantially”, and the like are used herein in the sense of “at, or nearly at, when given the manufacturing and material tolerances inherent in the stated circumstances” are used to prevent the unscrupulous infringer from unfairly taking advantage of the invention disclosure where exact or absolute figures are stated as an aid to understanding the invention.

“Machine direction” or MD means the length of a fabric in the direction in which it is produced. The term “cross machine direction” or CD means the width of fabric, i.e. a direction generally perpendicular to the MD.

“Particle,” “particles,” “particulate,” “particulates” and the like, refer to a material that is generally in the form of discrete units. The particles can include granules, pulverulents, powders or spheres. Thus, the particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for use herein. The use of “particle” or “particulate” may also describe an agglomeration including more than one particle, particulate or the like.

Description

### Representative Process for Making a First Interbonded Fibrous Layer of the Present Invention

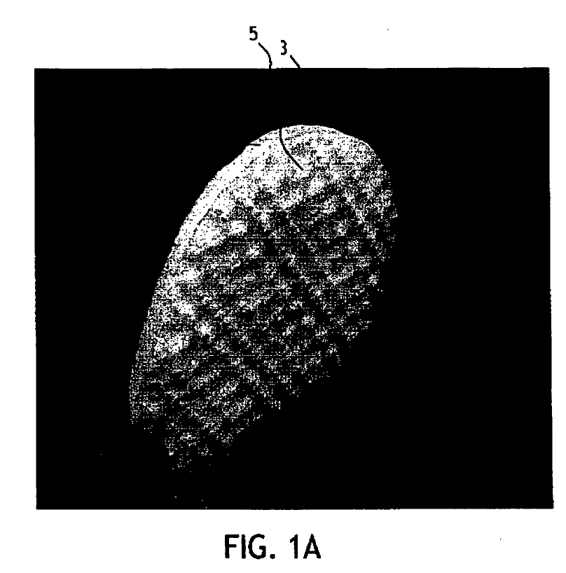

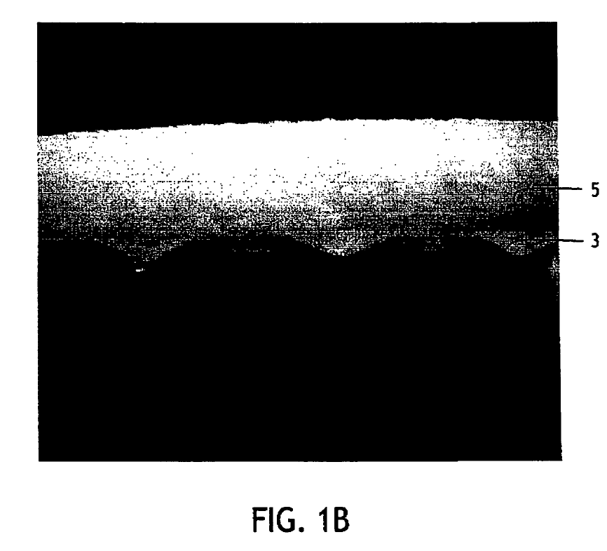

FIG. 2 is a representative schematic view of a process for forming a first interbonded fibrous layer of the present invention. The process is generally represented by reference numeral 100. In forming the first interbonded fibrous layer and any optional reinforcing strands which are attached to (at least in part) the first interbonded fibrous layer, pellets or chips, etc. (not shown) of an extrudable polymer are introduced into pellet hoppers 102 and 104 of extruders 106 and 108. (Note: FIG. 3 is a representative view of a process for forming a second interbonded fibrous layer of the present invention, and is described in more detail below.)

Each extruder has an extrusion screw (not shown) which is driven by a conventional drive motor (not shown). As the polymer advances through the extruder, due to rotation of the extrusion screw by the drive motor, it is progressively heated to a molten state. Heating the polymer to the molten state may be accomplished in a plurality of discrete steps with its temperature being gradually elevated as it advances through discrete heating zones of the extruder 106 toward a meltblowing die 110 and extruder 108 toward a continuous strand forming means 112 (i.e., a reinforcing strand forming means for the optional reinforcing strand(s)). The meltblowing die 110 and the continuous strand forming means 112 may be yet another heating zone where the temperature of the thermoplastic resin is maintained at an elevated level for extrusion. Heating of the various zones of the extruders 106 and 108 and the meltblowing die 110 and the continuous strand forming means 112 may be achieved by any of a variety of conventional heating arrangements (not shown).

The optional reinforcing strand component may be formed utilizing a variety of extrusion techniques. For example, the reinforcing strands may be formed utilizing one or more conventional meltblowing die arrangements which have been modified to remove the heated gas stream (i.e., the primary air stream) which flows generally in the same direction as that of the extruded strands to attenuate the extruded strands. This modified meltblowing die arrangement 112 usually extends across a collecting surface or support 114 in a direction which is substantially transverse to the direction of movement of the collecting surface or support 114. The modified die arrangement 112 includes a linear array 116 of small diameter capillaries aligned along the transverse extent of the die with the transverse extent of the die being approximately as long as the desired width of the parallel rows (or other alignment) of any optional reinforcing strands which are to be produced. That is, the transverse dimension of the die is the dimension which is defined by the linear array of die capillaries. The diameter of the capillaries may be on the order of from about 0.01 inches to about 0.02 inches, or, for example, from about 0.0145 to about 0.018 inches. But larger diameter capillaries may be used to enhance the exfoliating characteristics of the first interbonded fibrous layer, to reinforce the first interbonded fibrous layer, or both. Thus the reinforcing strands may be significantly larger (e.g., the reinforcing strands may be extruded through capillaries having a diameter of between about 0.020 inches and about 0.050 inches, or even larger). From about 0 to about 50 such capillaries will be provided per linear inch of die face. Typically, the length of the capillaries will be from about 0.05 inches to about 0.20 inches, for example, about 0.113 inches to about 0.14 inches long. A meltblowing die can extend from about 10 inches to about 60 or more inches in length in the transverse direction.

Since the heated gas stream (i.e., the primary air stream) which flows past the die tip is greatly reduced or absent, it may be desirable to insulate the die tip or provide heating elements to ensure that the extruded polymer remains molten and flowable while in the die tip. Polymer is extruded from the array 116 of capillaries in the modified die 112 to create any optional, extruded reinforcing strands 118.

The optional extruded reinforcing strands 118 have an initial velocity as they leave the array 116 of capillaries in the modified die 112. These strands 118 are deposited upon a surface 114 which should be moving at least at the same velocity as the initial velocity of the strands 118. This surface or support 114 is an endless belt conventionally driven by rollers 120. In the depicted representative embodiment, the strands 118 are deposited in substantially parallel alignment on the surface of the endless belt 114 which is rotating as indicated by the arrow 122 in FIG. 2. Vacuum boxes (not shown) may be used to assist in retention of the matrix on the surface of the belt 114. The tip of the die 112 should be as close as practical to the surface of the belt 114 upon which the reinforcing strands 118 are collected. For example, this forming distance may be from about 1 inch to about 10 inches. Desirably, this distance is from about 1 inch to about 8 inches.

It may be desirable to have the surface 114 moving at a speed that is much greater than the initial velocity of the reinforcing strands 118 in order to enhance the alignment of the strands 118 into substantially parallel rows and/or elongate the filaments 118 so they achieve a desired diameter. For example, alignment of the strands 118 may be enhanced by having the surface 114 move at a velocity from about 2 to about 10 times greater than the initial velocity of the strands 118. Even greater speed differentials may be used if desired. While different factors will affect the particular choice of velocity for the surface 114, it will typically be from about four to about ten times faster than the initial velocity of the reinforcing strands 118.

Desirably, the optional reinforcing strands are formed at a density per inch of width of material which corresponds generally to the density of capillaries on the die face. For example, the strand density per inch of width of material may range from 0 to about 120 such filaments per inch width of material. Typically, lower densities of filaments (e.g., 0-35 filaments per inch of width) may be achieved with only one strand forming die. Higher densities (e.g., 35-120 strands per inch of width) may be achieved with multiple banks of strand-forming equipment.

In the representative version of FIG. 2, the first interbonded fibrous layer is meltblown fiber. Here the meltblown fiber component is formed utilizing a conventional meltblowing process represented by reference numeral 124. Meltblowing processes generally involve extruding a thermoplastic polymer resin through a plurality of small diameter capillaries of a meltblowing die as molten threads into a heated gas stream (the primary air stream) which is flowing generally in the same direction as that of the extruded threads so that the extruded threads are attenuated, i.e., drawn or extended, to reduce their diameter. Such meltblowing techniques and apparatus are discussed fully in U.S. Pat. No. 4,663,220, which is hereby incorporated by reference in its entirety in a manner consistent herewith.

In the meltblown die arrangement 110, the position of air plates which, in conjunction with a die portion define chambers and gaps, may be adjusted relative to the die portion to increase or decrease the width of the attenuating gas passageways so that the volume of attenuating gas passing through the air passageways during a given time period can be varied without varying the velocity of the attenuating gas. Generally speaking, lower attenuating gas velocities and wider air passageway gaps are generally preferred if substantially continuous meltblown fibers or microfibers are to be produced.

The two streams of attenuating gas converge to form a stream of gas which entrains and attenuates the molten threads, as they exit the orifices, into fibers, depending upon the degree of attenuation, microfibers, of a small diameter which is usually less than the diameter of the orifices. The air stream is generally referred to using the term “primary air” in the Examples section below. The gas-borne fibers or microfibers 126 are blown, by the action of the attenuating gas, onto a collecting arrangement which, in the embodiment illustrated in FIG. 2, is the endless belt 114 (which optionally carries the reinforcing strand in substantially parallel alignment). The fibers or microfibers 126 are collected as a coherent matrix of fibers on the surface of support 114 (or, if present, the reinforcing strands 118) which is rotating as indicated by the arrow 122 in FIG. 2. If desired, the meltblown fibers or microfibers 126 may be collected on the endless belt 114 at numerous impingement angles. A vacuum box 140 is used to draw the meltblown fibers into the openings 142 in the endless belt or support 114. By adjusting process parameters (e.g., amount of vacuum; temperature at which meltblown fibers exit the orifices), the interbonded fibrous layer is drawn into the openings in the support 114 so that shaped discontinuities are formed in the interbonded fibrous layer itself. I.e., the shaped discontinuities in the interbonded fibrous layer correspond to the openings in support 114. It should be noted that this forming process does not create the amount of waste inherent in cutting holes or other openings directly in the interbonded fibrous layer (if the depressions/valleys are perforated—i.e., have openings by virtue of the fiber drawing apart and separating within the opening in the support). In the present invention, the meltblown fibers proximate to (i.e., over or near) openings 142 are further attenuated by the action of the vacuum drawing the fiber into the openings. If desired, by selecting the aforementioned process parameters a portion of the attenuated fiber within the openings separate, thereby forming perforations or openings at the tip of any projection emanating from the surface of the interbonded fibrous layer (and contiguous with the shaped opening in the interbonded fibrous layer itself).

It should be noted that the depicted openings 142 in the support 114 in FIG. 3 are representative. The shape, size, number, and placement of such openings can be varied. For example, the openings in the belt may be rectangles, squares, triangles, ovals, stars, crosses, pentagons, hexagons, octagons, other such geometric shapes, and various combinations thereof. Furthermore, the openings, die cut or otherwise, may be more complex, and in fact may depict various recognizable living or non-living objects. For example, an opening defining the shape of a teddy bear might be used. Or an opening defining the shape of a tulip, air plane, rocket, or any number of other such objects might be used. Or, as mentioned above, a company's logo, tradename, or trademark might be introduced to the support 114 so that the corresponding image is introduced to the first interbonded fibrous layer.

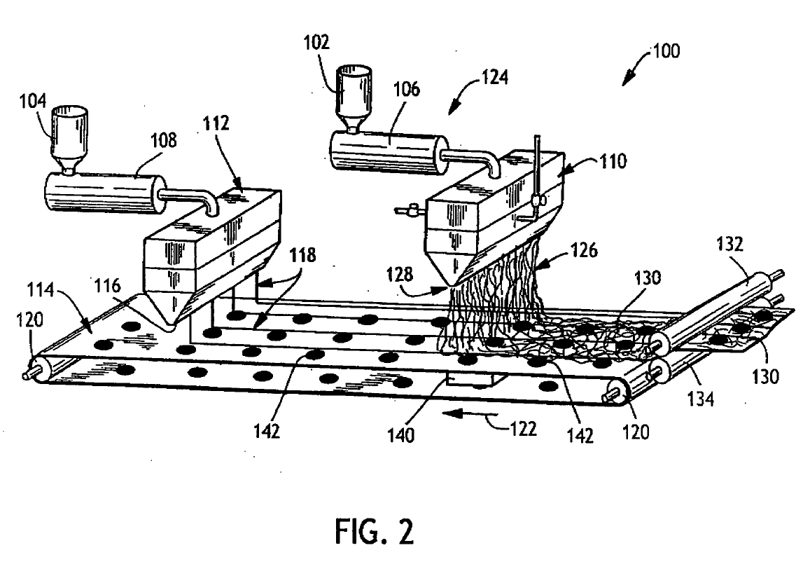

It should also be noted that the surface of the belt itself may be textured. Examples of various textured surfaces include a pebbled surface; a surface having the appearance of a molded screen—with individual strands interleaved with one another; a surface having the appearance of a lattice with diamond-shaped openings; etc. Furthermore; the textured surface may have a complex surface topography, with multiple tiers. The thickness of the belt may be varied to accommodate the selected texture on the surface of the belt and the selected openings in the belt. A few representative versions of such textures are depicted in FIGS. 4, 4A, 5, 5A, 6, 6A, 7, and 7A.

FIG. 4 illustrates in, greater detail and in perspective view one forming surface which can be used as belt 114 in FIG. 3. As shown, the surface in this case is a flat belt 160 having cone-shaped pins 162 which are disposed outwardly from the surface. In this embodiment belt 160 also contains openings 164. FIG. 4A shows the forming surface of FIG. 4 in cross-section taken along lines 4A-4A. The forming surface in FIG. 4 could be used without the cone-shaped pins 162, and could further include different textures or surface topographies between the openings 164. As noted above, the openings may be of a variety of shapes other than circles, and the placement of these openings can be varied as desired. Although in the representative embodiment depicted in FIGS. 4 and 4A the openings have a uniform diameter through the thickness of the belt, the openings in the belt may be fashioned to have a changing diameter through the thickness of the belt.

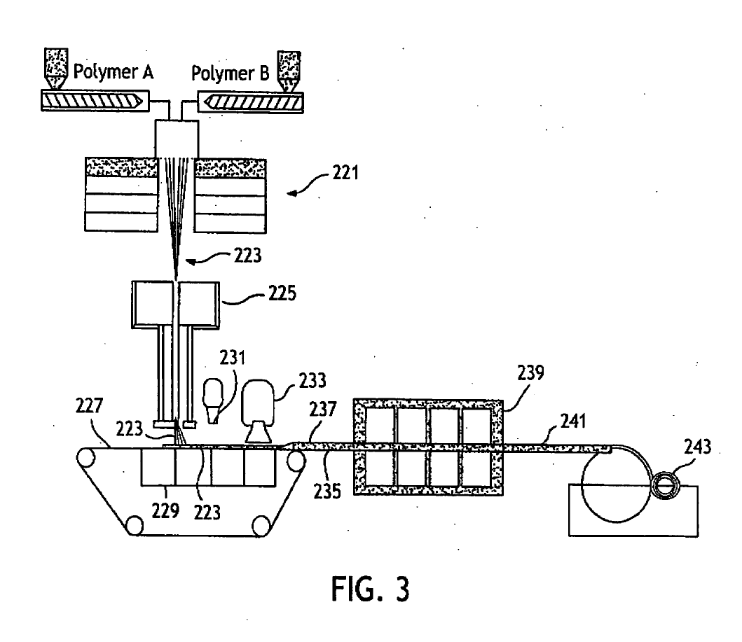

FIG. 5 is a view of an alternative forming surface 168 which, in this case, has pins 170 in the shape of truncated cones extending outwardly and openings 172. FIG. 5A is a cross-section of the surface of FIG. 5 taken along lines 5A-5A. The forming surface in FIG. 5 could be used without the cone-shaped pins 170, and could further include different textures or surface topographies between the openings 172. Also, if used, the pins could be further truncated to varying degrees short of total elimination of the pins. As noted above, the openings may be of a variety of shapes other than circles, and the placement of these openings can be varied as desired. Although in the representative embodiment depicted in FIGS. 5 and 5A the openings have a uniform diameter through the thickness of the belt, the openings in the belt may be fashioned to have a changing diameter through the thickness of the belt.

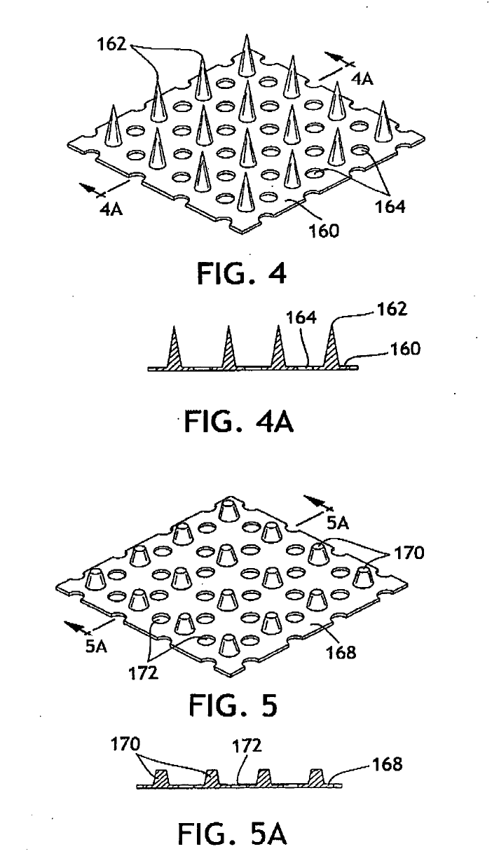

FIGS. 6 and 6A are views like FIGS. 4 and 4A illustrating yet other forming surfaces 178 having domes 180 at the surface of the belt.

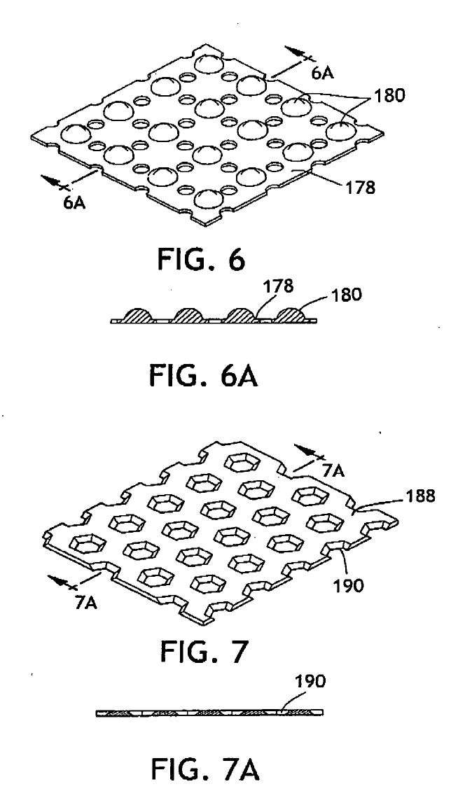

FIG. 7 illustrates an alternative belt configuration 188, in this case comprising hexagonal openings 190, useful in making an interbonded fibrous layer of the present invention, and FIG. 7A shows the belt of FIG. 7 in cross-section taken along lines 7A-7A. As noted earlier, openings need not have a uniform cross-section through the thickness of the belt. FIG. 7A shows that the interior surfaces of the hexagon slope inward to the center of the hexagon itself. Openings also may have multiple tiers through the thickness of the belt. I.e., the inner diameter (or other distance depending on the shape of the opening) may change in a step-wise fashion through the thickness of the belt (rather than in a monotonically increasing or decreasing fashion).

Vacuum boxes, such as that identified in the drawing by numeral 140, may be used to assist generally in retention of the matrix on the surface of the belt 114. Typically the tip 128 of the die 110 is from about 6 inches to about 14 inches from the surface of the belt 114 upon which the fibers are collected. The entangled fibers or microfibers 126 autogenously bond to each other and, if they are present, at least a portion of the reinforcing strands 118 because the fibers or microfibers 124 are still somewhat tacky or molten while they are deposited on the optional reinforcing strands 118, thereby forming the substrate 130.

At this point, it may be desirable to lightly calender the first interbonded fibrous layer in order to enhance the autogenous bonding. This optional calendering step may be accomplished with a pair of patterned or un-patterned pinch rollers 132 and 134 under sufficient pressure (and temperature, if desired) to help facilitate autogenous bonding between the fibers making up the first interbonded fibrous layer (here a meltblown layer), and any optional reinforcing strands.

As discussed above, the optional reinforcing strands and first interbonded fibrous layer are deposited on a moving surface. In one embodiment of the invention, meltblown fibers are formed directly on top of the optional extruded reinforcing strands. This is achieved by passing the strands and support under equipment that produces the interbonded fibrous layer (meltblown material in the version of the process depicted in FIG. 2). Alternatively, the first interbonded fibrous layer, such as a meltblown material, may be deposited on a surface and substantially parallel rows (or other arrangement) of the optional reinforcing strands may be formed directly upon the first interbonded fibrous layer. Various combinations of strand-forming and fiber-forming equipment may be set up to produce different types of substrates. For example, the substrate may contain alternating layers of reinforcing strands and interbonded fibrous layers. Several dies for forming interbonded fibrous layers or creating reinforcing strands may also be arranged in series to provide superposed layers of fibers or strands. And, of course, the first interbonded fibrous layer may be made without reinforcing strands (e.g., comprised of a meltblown material without reinforcing strands).

The location of the means for forming the optional reinforcing strands relative to the location of the means for forming the first interbonded fibrous layer may be selected (taking into consideration the range of velocities at which support 114 moves) to obtain desired time intervals between the time at which the optional reinforcing strands are extruded and the time at which the first interbonded fibrous layer contacts the reinforcing strands (or vice versa, if the first interbonded fibrous layer is formed first, and the reinforcing strands are extruded onto the first interbonded fibrous layer). Typically the time interval will allow for the reinforcing strands, the first interbonded fibrous layer, or both, to be somewhat tacky and to be capable of autogenous bonding. Note, however, that an adhesive could be applied to the reinforcing strands, the interbonded fibrous layer, or both to promote bonding.

As noted above, the invention contemplates the possibility of multiple banks of dies for forming the interbonded fibrous layer, the reinforcing strands, or both. Furthermore, the individual capillaries within a linear array of said capillaries; between multiple banks of linear arrays of capillaries; or both, may be of different sizes. Also, the operating parameters for a given linear array of capillaries (e.g., temperature at which the molten polymer exits the capillaries; velocity and/or temperature of any air flow used to carry and/or attenuate the exiting fiber or strand; etc.) may be different across said linear array; between multiple banks of linear arrays of capillaries; or both.

### Representative Materials with which the Reinforcing Strand and/or First Interbonded Fibrous Layer may be Made

The first interbonded fibrous layer and any optional reinforcing strands may be made from any material which may be manufactured into such fibrous layer and strands. For those personal-care appliances requiring or benefiting from elastomeric characteristics, the substrate may be made using suitable elastomeric fiber-forming resins or blends containing the same for the interbonded fibrous layer; and any suitable elastomeric strand-forming resins or blends containing the same may be utilized for the reinforcing strands. The fibers and filaments may be formed from the same or different elastomeric resin.

For example, the interbonded fibrous layer and/or the reinforcing strands may be made from block copolymers having the general formula A-B-A′ where A and A′ are each a thermoplastic polymer endblock which contains a styrenic moiety such as a poly (vinyl arene) and where B is an elastomeric polymer midblock such as a conjugated diene or a lower alkene polymer. The block copolymers may be, for example, (polystyrene/poly(ethylene-butylene)/polystyrene) block copolymers available from the Shell Chemical Company under the trademark KRATON G. One such block copolymer may be, for example, KRATON G-1657.

Other exemplary materials which may be used include polyurethane materials such as, for example, those available under the trademark ESTANE from B. F. Goodrich & Co., polyamide materials such as, for example, those available under the trademark PEBAX from the Rilsan Company, and polyester materials such as, for example, those available under the trade designation Hytrel from E. I. DuPont De Nemours & Company. Formation of meltblown fibers from polyester materials is disclosed in, for example, U.S. Pat. No. 4,741,949 to Morman et al., which is hereby incorporated by reference in its entirety in a manner consistent herewith. Useful polymers also include, for example, copolymers of ethylene and at least one vinyl monomer such as, for example, vinyl acetates, unsaturated aliphatic monocarboxylic acids, and esters of such monocarboxylic acids. The copolymers and formation of meltblown fibers from those copolymers are disclosed in, for example, U.S. Pat. No. 4,803,117.

Processing aids may be added to the polymer. For example, a polyolefin may be blended with the polymer (e.g., the A-B-A elastomeric block copolymer) to improve the processability of the composition. The polyolefin must be one which, when so blended and subjected to an appropriate combination elevated pressure and elevated temperature conditions, extrudable, in blended form, with the polymer. Useful blending polyolefin materials include, for example, polyethylene, polypropylene and polybutene, including ethylene copolymers, propylene copolymers and butene copolymers. A particularly useful polyethylene may be obtained from the U.S.I. Chemical Company under the trade designation Petrothene NA 601 (also referred to herein as PE NA 601 or polyethylene NA 601). Two or more of the polyolefins may be utilized. Extrudable blends of polymers and polyolefins are disclosed in, for example, previously referenced U.S. Pat. No. 4,663, 220.

The first interbonded fibrous layer and/or the reinforcing strands may have some tackiness adhesiveness to enhance autogenous bonding. For example, the polymer itself may be tacky when formed into fibers and/or strands or, alternatively, a compatible tackifying resin may be added to the extrudable compositions described above to provide tackified fibers and/or strands that autogenously bond. In regard to the tackifying resins and tackified extrudable compositions, note the resins and compositions as disclosed in U.S. Pat. No. 4,787,699, hereby incorporated by reference in its entirety in a manner consistent herewith.

Any tackifier resin can be used which is compatible with the polymer and can withstand the processing (e.g., extrusion) temperatures. If the polymer (e.g., A-B-A elastomeric block copolymer) is blended with processing aids such as, for example, polyolefins or extending oils, the tackifier resin should also be compatible with those processing aids. Generally, hydrogenated hydrocarbon resins are preferred tackifying resins, because of their better temperature stability. REGALREZ and ARKON series tackifiers are examples of hydrogenated hydrocarbon resins. ZONATAK 501 lite is an example of a terpene hydrocarbon. REGALREZ hydrocarbon resins are available from Hercules incorporated. ARKON series resins are available from Arakawa Chemical (U.S.A.) Incorporated. Of course, the present invention is not limited to use of such three tackifying resins, and other tackifying resins which are compatible with the other components of the composition and can withstand the processing temperatures, can also be used.

Typically, the blend used to form the reinforcing strands and fibers for the interbonded fibrous layer include, for example, from about 40 to about 80 percent by weight polymer, from about 5 to about 40 percent polyolefin and from about 5 to about 40 percent resin tackifier. For example, a particularly useful composition included, by weight, about 61 to about 65 percent KRATON G-1657, about 17 to about 23 percent polyethylene NA 601, and about 15 to about 20 percent REGALREZ 1126.

The first interbonded fibrous layer component of a substrate of the present invention may be a mixture of elastic and nonelastic fibers or particulates. For an example of such a mixture, reference is made to U.S. Pat. No. 4,209,563, which is hereby incorporated by reference in its entirety in a manner consistent herewith, in which elastomeric and non-elastomeric fibers are commingled to form a single coherent web of randomly dispersed fibers. Another example of such an composite web would be one made by a technique such as disclosed in previously referenced U.S. Pat. No. 4,741,949. That patent discloses an elastic nonwoven material which includes a mixture of meltblown thermoplastic fibers and other materials. The fibers and other materials are combined in the gas stream in which the meltblown fibers are borne so that an intimate entangled commingling of meltblown fibers and other materials, e.g., wood pulp, staple fibers or particulates such as, for example, activated charcoal, clays, starches, or hydrocolloid (hydrogel) particulates commonly referred to as super-absorbents occurs prior to collection of the fibers upon a collecting device to form a coherent web of randomly dispersed fibers.

To give the substrate, and any personal-care appliances made therefrom, increased wet resilience, strength, and/or exfoliating character, the first interbonded fibrous layer and any optional reinforcing strands may be made from a polyolefin such as polypropylene. Particularly suitable polymers for forming the reinforcing fiber include polypropylene and copolymers of polypropylene and ethylene. Other polymers useful in the manufacture of reinforcing strand (and/or the interbonded fibrous layer) may further include thermoplastic polymers like polyolefins, polyesters and polyamides. Elastic polymers rhay also be used and include block copolymers such as polyurethanes, copolyether esters, polyamide polyether block copolymers, ethylene vinyl acetates (EVA), block copolymers having the general formula A-B-A′ or A-B like copoly(styrene/ethylene-butylene), styrene-poly(ethylene-propylene)-styrene, styrene-poly(ethylene-butylene)-styrene, (polystyrene/poly(ethylene-butylene)/polystyrene, poly(styrene/ethylene-butylene/styrene) and the like.

Polyolefins using single site catalysts, sometimes referred to as metallocene catalysts, may also be used to make the interbonded fibrous layer and/or the reinforcing strands. Many polyolefins are available for fiber production, for example polyethylenes such as Dow Chemical's ASPUN7 6811A linear low density polyethylene, 2553 LLDPE and 25355 and 12350 high density polyethylene are such suitable polymers. The polyethylenes have melt flow rates, respectively, of about 26, 40, 25 and 12. Fiber forming polypropylenes include Exxon Chemical Company's 3155 polypropylene and Montell Chemical Co.'s PF-304 and/or PF-015. Many other polyolefins are commercially available.

Biodegradable polymers are also available for interbonded fiber and reinforcing strand production and suitable polymers include polylactic acid (PLA) and a blend of BIONOLLE, adipic acid and UNITHOX (BAU). PLA is not a blend but a pure polymer like polypropylene. BAU represents a blend of BIONOLLE, adipic acid, and UNITHOX at different percentages. Typically, the blend for staple fiber is 44.1 percent BIONOLLE 1020, 44.1 percent BIONOLLE 3020, 9.8 percent adipic acid and 2 percent UNITHOX 480, though spunbond BAU fibers typically use about 15 percent adipic acid. BIONOLLE 1020 is polybutylene succinate, BIONOLLE 3020 is polybutylene succinate adipate copolymer, and UNITHOX 480 is an ethoxylated alcohol. BIONOLLE is a trademark of Showa Highpolymer Co. of Japan. UNITHOX is a trademark of Baker Petrolite which is a subsidiary of Baker Hughes International.

Polypropylene, and other such polymeric materials, generally make for a stiffer, stronger fiber, especially if, the fiber is made with a larger diameter. Furthermore, the polymeric materials from which any optional reinforcing strand is made can be selected so that the reinforcing strands soften at a temperature higher than the temperature at which the first interbonded fibrous layer softens. For those embodiments where any optional reinforcing strands are extruded over openings in support 114 (see FIG. 2), selection of the material, or materials of construction, of the reinforcing strands such that the strands have a softening point higher than that of the first interbonded fibrous layer can help ensure that the reinforcing strands are not pulled into the openings 140 when a vacuum 142 is applied. Alternatively, the location of the small diameter capillaries along the transverse dimension of the die may be selected such that the reinforcing strands are not extruded over openings in the support. And, of course, the materials of construction of any optional reinforcing strand may be selected so that any reinforcing strand near or over openings 140 is drawn into said opening.

### Representative Process for Making a Second Interbonded Fibrous Layer of the Present Invention

As stated above, FIG. 3 is a schematic diagram illustrating methods and apparatus of this invention for producing a second interbonded fibrous layer comprising a high-loft, low-density material. In this case the second interbonded fibrous layer is made by producing crimpable bicomponent substantially continuous fibers of A/B morphology, i.e., a bilateral configuration, generally side by side or eccentric sheath/core, and causing them to crimp in an unrestrained environment.

As shown in FIG. 3, two polymers A and B are spunbond with known thermoplastic fiber spinning apparatus 221 to form bicomponent, or A/B, morphology fibers 223. The fibers 223 are then traversed through a fiber draw unit (FDU) 225. According to one embodiment of the present invention, unlike the standard practice in the art, the FDU is not heated, but is left at ambient temperature. The fibers 223 are left in a substantially continuous state and are deposited on a moving forming support 227. Deposition of the fibers is aided by an under-wire vacuum supplied by a negative air pressure unit, or below wire exhaust, 229.

The fibers 223 are then heated by traversal under one of a hot air knife (HAK) 231 or hot air diffuser 233 , which are both shown in the figure but will be appreciated to be used in the alternative under normal circumstances. A conventional hot air knife includes a mandrel with a slot that blows a jet of hot air onto the nonwoven web surface. Such hot air knives are taught, for example, by U.S. Pat. No. 5,707,468 to Arnold, et al. The hot air diffuser 233 is an alternative which operates in a similar manner but with lower air velocity over a greater surface area and thus uses correspondingly lower air temperatures. The group, or layer, of fibers may receive an external skin melting or a small degree of nonfunctional bonding during this traversal through the first heating zone. “Nonfunctionally bonded” is a bonding sufficient only to hold the fibers in place for processing according to the method herein but so light as to not hold the fibers together were they to be manipulated manually. Such bonding may be incidental or eliminated altogether if desirable.

The fibers are then passed out of the first heating zone of the hot air knife 231 or hot air diffuser 233 to a second wire 235 where the fibers continue to cool and where the below wire exhaust 229 is removed so as to not disrupt crimping. As the fibers cool they will crimp in the z-direction, or out of the plane of the web, and form a high loft, low density nonwoven web 237. The web 237 is then transported to a through air bonding (TAB) unit 239 to set, or fix, the web at a desired degree of loft and density. Alternatively, the through air bonding (TAB) unit 239 can be zoned to provide a first heating zone in place of the hot air knife 231 or hot air diffuser 233, followed by a cooling zone, which is in turn followed by a second heating zone sufficient to fix the web. The fixed web 241 can then be collected on a winding roll 243 or the like for later use in constructing a personal-care appliance of the present invention.

In accordance with one embodiment of this invention, the substantially continuous fibers in the second interbonded fibrous layer are bicomponent fibers. Webs of the present invention may contain a single denier structure (i.e., one fiber size) or a mixed denier structure (i.e., a plurality of fiber sizes). Particularly suitable polymers for forming the structural component of suitable bicomponent fibers include polypropylene and copolymers of polypropylene and ethylene, and particularly suitable polymers for the adhesive component of the bicomponent fibers includes polyethylene, more particularly linear low density polyethylene, and high density polyethylene. In addition, the adhesive component may contain additives for enhancing the crimpability and/or lowering the bonding temperature of the fibers, as well as enhancing the abrasion resistance, strength and softness of the resulting webs. A particularly suitable bicomponent polyethylene/polypropylene fiber for processing according to the present invention is known as PRISM. A description of PRISM is disclosed in U.S. Pat. No. 5,336,552 to Strack et al. Webs made according to the present invention may further contain fibers having resins alternative to PP/PE, such as, without limitation: PET, Copoly-PP+3% PE, PLA, PTT, Nylon, PBT, etc. Fibers may be of various alternative shapes and symmetries including Pentaloble, Tri-T, Hollow, Ribbon, X, Y, H, and asymmetric cross sections.

Polymers useful in the manufacture of second interbonded fibrous layer may further include thermoplastic polymers like polyolefins, polyesters and polyamides. Elastic polymers may also be used and include block copolymers such as polyurethanes, copolyether esters, polyamide polyether block copolymers, ethylene vinyl acetates (EVA), block copolymers having the general formula A-B-A′ or A-B like copoly(styrene/ethylene-butylene), styrene-poly(ethylene-propylene)-styrene, styrene-poly(ethylene-butylene)-styrene, (polystyrene/poly(ethylene-butylene)/polystyrene, poly(styrene/ethylene-butylene/styrene) and the like.

Polyolefins using single site catalysts, sometimes referred to as metallocene catalysts, may also be used. Many polyolefins are available for fiber production, for example polyethylenes such as Dow Chemical's ASPUN7 6811A linear low density polyethylene, 2553 LLDPE and 25355 and 12350 high density polyethylene are such suitable polymers. The polyethylenes have melt flow rates, respectively, of about 26, 40, 25 and 12. Fiber forming polypropylenes include Exxon Chemical Company's 3155 polypropylene and Montell Chemical Co.'s PF-304. Many other polyolefins are commercially available.

Biodegradable polymers are also available for fiber production and suitable polymers include polylactic acid (PLA) and a blend of BIONOLLE, adipic acid and UNITHOX (BAU). PLA is not a blend but a pure polymer like polypropylene. BAU represents a blend of BIONOLLE, adipic acid, and UNITHOX at different percentages. Typically, the blend for staple fiber is 44.1 percent BIONOLLE 1020, 44.1 percent BIONOLLE 3020, 9.8 percent adipic acid and 2 percent UNITHOX 480, though spunbond BAU fibers typically use about 15 percent adipic acid. BIONOLLE 1020 is polybutylene succinate, BIONOLLE 3020 is polybutylene succinate adipate copolymer, and UNITHOX 480 is an ethoxylated alcohol. BIONOLLE is a trademark of Showa Highpolymer Co. of Japan. UNITHOX is a trademark of Baker Petrolite which is a subsidiary of Baker Hughes International. It should be noted that these biodegradable polymers are hydrophilic and so are preferably not used for the surface of the inventive intake system materials.

Per the above, the crimpable bicomponent fiber is heated by the HAK 231, hot air diffuser 233 or zoned TAB (not shown) in the first heating zone to a temperature where the polyethylene crystalline regions start to relax their oriented molecular chains and may begin melting. Typical air temperature used to induce crimp have ranged from about 110-260 degrees F. This temperature range represents temperatures of submelting degree which merely relax the molecular chain up through melting temperatures for the polymers. The heat of the air stream from the HAK 231 may be made higher due to the short dwell time of the fibers through its narrow heating zone. Further, when heat is applied to the oriented molecular chains of the fibers, the molecular chain mobility increases. Rather that being oriented, the chains prefer to relax in a random state. Therefore, the chains bend and fold causing additional shrinkage. Heat to the web may be applied by hot air, IR lamp, microwave or any other heat source that can heat the semi-crystalline regions of the polyethylene to relaxation.

Then the web passes through a cool zone that reduces the temperature of the polymer below its crystallization temperature. Since polyethylene is a semi-crystalline material, the polyethylene chains recrystallize upon cooling causing the polyethylene to shrink. This shrinkage induce a force on one side of the side-by-side fiber that allows it to crimp or coil if there are no other major forces restricting the fibers from moving freely in any direction. By using the cold FDU, the fibers are constructed so that they do not crimp in a tight helical fashion normal for fibers processed through a normal hot FDU. Instead, the fibers more loosely and randomly crimp, thereby imparting more z-direction loft to the fibers.

Factors that can affect the amount and type of crimp include the dwell time.of the web under the heat of the first heating zone. Other factors affecting crimp can include material properties such as fiber denier, polymer type, cross sectional shape and basis weight. Restricting the fibers with either a vacuum, blowing air, or bonding will also affect the amount of crimp and thus the loft, or bulk, desired to be achieved in the high loft, low density webs of the present invention. Therefore, as the fibers enter the cooling zone, no vacuum is applied to hold the fibers to the forming wire 227 or second wire 235. Blowing air is likewise controlled or eliminated in the cooling zone to the extent practical or desired.

The fibers may be deposited on the forming wire with a high degree of MD orientation as controlled by the amount of under-wire vacuum, the FDU pressure, and the forming height from the FDU to the wire surface. A high degree of MD orientation may be used to induce very high loft into the web, as further explained below. Further, dependent upon certain fiber and processing parameters, the air jet of the FDU will exhibit a natural frequency which may aid in the producing of certain morphological characteristics such as shingling effects into the loft of the web.

According to the exemplary embodiment of FIG. 3, wherein the fibers 223 are heated by air flow in the first heating zone and passed by the forming wire 227 to the second wire 235, several crimping mechanisms are believed to take place to aid in the lofting of the fibers, including, without being bound by theory: the below-wire exhaust will cool the web by drawing surrounding air through it which prevent bonding but restricts formation of loft; as the web is transferred out of the vacuum zone to the second wire, the vacuum force is removed and the unconstrained fibers are free to crimp; mechanically, MD surface layer shrinkage of a highly MD oriented surface layer may cause the surface fibers to buckle; mechanical shearing will be induced because the highly MD oriented surface shirring and bonds will leave subsurface fibers to continue shearing thereby creating loft by inducing shingling of the layers; a mechanical buckling pattern may be produced at the natural frequency of the FDU jet which will cause the heated fibers to loft in the same frequency; mechanical forces are created as fibers release from the forming wire 227 when leaving the vacuum area and then are briefly pulled back towards the vacuum unit 229; and a triboelectric (frictional) static charge is built up on the web and causes the fibers to repel each other allowing further loft within the web.

Additional detail regarding forming of the second interbonded fibrous layer using the exemplary process described above may be found in U.S. Patent Publication Number 2005/0098256 A1, entitled “High Loft Low Density Nonwoven Webs of Crimped Filaments and Methods of Making Same” and listing Polanco, Braulio et al. as inventors. This U.S. patent publication is hereby incorporated by reference in its entirety in a manner consistent herewith.

### Representative Two-Sided Personal-Care Appliance

The first and second interbonded fibrous layers may be combined in a number of ways. For example, an adhesive (e.g., a hot-melt adhesive, a blend of atactic and isotactic polyolefins, or other such materials) may be applied to a surface of either or both layers before joining the two layers together. The adhesive may be sprayed, coated, printed, or otherwise associated with the surface of one or both layers. Alternatively, energy in the form of, for example, heat can be directed to one or both surfaces thereby softening or otherwise making tacky that fiber at or near the heated surface. The two layers can then be joined at said heated surface(s), with fiber at both surfaces fusing or adhering to one another. After the application of an adhesive or the input of energy, the resulting laminate can be directed through a nip between two rolls to assist in attaching one layer to the other.

The first and second interbonded fibrous layers may be made and attached to one another in a single operating line. Alternatively, each layer may be made separately, with each layer then being wound up to form a roll. These rolls could then be placed on reels and systematically unwound such that the layers are joined together at their surfaces, whether adhesively, through the input of energy, or both. These layers may be joined at the same geographic location where they are made. Or one or both layers may be made at one or more geographic locations, and then shipped to another geographic location where the layers are joined.

Generally the two-sided personal-care appliance will be in a form and shape adapted for use by a consumer, purchaser, or user of the appliance. Thus the appliance may be a rectangle, square, oval, or the like. Alternatively the appliance may be in the shape of an egg, star, hexagon, octagon, or other such similar shape. Generally any shape may be selected, so long as the appliance may be used for cleaning and/or moisturizing and/or exfoliating, stimulating, or gently abrading skin or tissue.

The selected shapes may be produced by cutting or otherwise obtaining the shapes from each of the interbonded fibrous layers, and then joining or attaching these cut or otherwise obtained shapes to one another. Alternatively, the first and second interbonded fibrous layers may be joined to one another, and, after the combination has been formed, the desired shape is produced by cutting or otherwise obtaining the shape from the combination.

The present invention also contemplates one or more layers interposed between the first interbonded fibrous layer and the second interbonded fibrous layer. Basically any configuration or mode of construction may be used so long as the resulting appliance comprises a first interbonded fibrous layer and second interbonded fibrous layer, each having the structure and characteristics recited elsewhere in this document.

A cleaning and/or moisturizing composition may be introduced to either or both of the fibrous layers, before or after the making of the laminate, or before or after the cutting or obtaining of the desired shape of the personal-care appliance.

### Representative Cleaning Compositions that may be Deposited on a Two-Side Personal-Care Appliance of the Present Invention

Cleaning compositions that may be deposited on or otherwise associated with two-sided personal-care appliances of the present invention include soaps, skin lotions, colognes, sunscreens, shampoos, gels, bodywashes, and the like. Such compositions may be in solid, liquid, gel, foam, or other forms. Such compositions may also include, or be, moisturizing agents or formulations.

Many cleaning compositions contain similar core ingredients, such as water and surfactants. They may also contain oils, detergents, emulsifiers, film formers, waxes, perfumes, preservatives, emollients, solvents, thickeners, humectants, chelating agents, stabilizers, pH adjusters, and so forth. In U.S. Pat. No. 3,658,985, for example, an anionic based composition contains a minor amount of a fatty acid alkanolamide. U.S. Pat. No. 3,769,398 discloses a betaine-based composition containing minor amounts of nonionic surfactants. U.S. Pat. No. 4,329,335 also discloses a composition containing a betaine surfactant as the major ingredient and minor amounts of a nonionic surfactant and of a fatty acid mono- or di-ethanolamide. U.S. Pat. No. 4, 259,204 discloses a composition comprising 0.8 to 20% by weight of an anionic phosphoric acid ester and one additional surfactant which may be either anionic, amphoteric, or nonionic. U.S. Pat. No. 4,329,334 discloses an anionic amphoteric based composition containing a major amount of anionic surfactant and lesser amounts of a betaine and nonionic surfactants.

U.S. Pat. No. 3,935,129 discloses a liquid cleaning composition containing an alkali metal silicate, urea, glycerin, triethanolamine, an anionic detergent and a nonionic detergent. The silicate content determines the amount of anionic and/or nonionic detergent in the liquid cleaning composition. U.S. Pat. No. 4,129,515 discloses a liquid detergent comprising a mixture of substantially equal amounts of anionic and nonionic surfactants, alkanolamines and magnesium salts, and, optionally, zwitterionic surfactants as suds modifiers. U.S. Pat. No. 4, 224,195 discloses an aqueous detergent composition comprising a specific group of nonionic detergents, namely, an ethylene oxide of a secondary alcohol, a specific group of anionic detergents, namely, a sulfuric ester salt of an ethylene oxide adduct of a secondary alcohol, and an amphoteric surfactant which may be a betaine, wherein either the anionic or nonionic surfactant may be the major ingredient. Detergent compositions containing all nonionic surfactants are shown in U.S. Pat. Nos. 4,154,706 and 4,329,336. U.S. Pat. No. 4,013,787 discloses a piperazine based polymer in conditioning and shampoo compositions. U.S. Pat. No. 4,450,091 discloses high viscosity compositions containing a blend of an amphoteric betaine surfactant, a polyoxybutylenepolyoxyethylene nonionic detergent, an anionic surfactant, a fatty acid alkanolamide and a polyoxyalkylene glycol fatty ester. U.S. Pat. No. 4,595,526 describes a composition comprising a nonionic surfactant, a betaine surfactant, an anionic surfactant and a C12-C14 fatty acid mono-ethanolamide foam stabilizer. The contents of the patents discussed herein are hereby incorporated by reference as if set forth in their entirety and in a manner consistent herewith.

Further information on these ingredients may be obtained, for example, by reference to: Cosmetics & Toiletries, Vol. 102, No.3, Mar. 1987; Balsam, M. S., et al., editors, Cosmetics Science and Technology, 2nd edition, Vol.1, pp 27-104 and 179-222 Wiley-Interscience, New York, 1972, Vol. 104, pp 67-111, February 1989; Cosmetics & Toiletries, Vol.103, No.12, pp 100-129, Dec.1988, Nikitakis, J. M., editor, CTFA Cosmetic Ingredient Handbook, first edition, published by The Cosmetic, Toiletry and Fragrance Association, Inc., Washing-ton, D.C., 1988, Mukhtar, H, editor, Pharmacology of the Skin, CRC Press 1992; and Green, F J, The Sigma-Aldrich Handbook of Stains. Dyes and Indicators; Aldrich Chemical Company, Milwaukee Wis., 1991, the contents of which are hereby incorporated by reference as if set forth in their entirety and in a manner consistent herewith.

Exemplary materials that may be used in the practice of this invention further include but are not limited to those discussed in Cosmetic and Toiletry Formulations by Ernest W. Flick, ISBN 0-8155-1218-X, second edition, section XII (pages 707-744).

Other ingredients that may be included in a composition or formulation associated with a two-sided personal-care appliance of the present invention include emulsifiers, surfactants, viscosity modifiers, natural-moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, some combination thereof, and the like.

Such compositions and formulations may be applied to, on, or otherwise associated with the two-sided personal-care appliance in a variety of ways. For example, a composition or formulation may be injected into the second interbonded fibrous layer. Alternatively, the composition or formulation can be sprayed or coated onto the second interbonded fibrous layer. Also, a composition or formulation can be sprayed, coated, printed, extruded, or injected into or onto the personal-care appliance.

Typically soaps, compositions, or other formulations in liquid form will dissipate after 1 or 2 uses. In other words, a substantial portion of the initial quantity of soap, composition, or other formulation associated with the personal-care appliance will disassociate from the appliance during the first use. Disassociation will likely occur through the soap, composition, or formulation dissolving in, or otherwise being carried away by, water during use of the appliance. If the personal-care appliance is used a second time, then that portion of the soap, composition, or other formulation dissipated by the first use is not available for the second use. As stated above, after a few uses, the personal-care appliance has little or no soap, composition, or other formulation left. If the personal-care appliance is to be adapted for limited use by a user, dissipation of any associated soap, composition, or other formulation provides a signal to the user that the appliance may be disposed of. Manufacturers and/or distributors and/or retailers of the product may explicitly communicate to a purchaser or user that dissipation of the associated soap, composition, or other formulation signals that the appliance may be disposed of.

If the personal-care appliance is to be adapted for limited use, then the number of times the appliance may be used can be changed in a number of ways. For example, the physical properties of the soap, composition, or other formulation may be altered so that the rate at which the soap or other material dissolves or is carried away is altered. For example, the viscosity of the material may be increased. Or the hydrophilic/hydrophobic character of the composition may be changed. Alternatively, the soap, composition, or other formulation may be microencapsulated, with the microcapsules making available their contents after some external stimulus is provided (e.g., the microcapsules are broken by the application of an external force as would be present when a user is using the appliance or substrate; or the microcapsule is made using materials known to dissolve in water, with the rate of dissolution of the microcapsules selected so that the availability of the microencapsulated materials during use is extended over the desired number of uses). In another approach, the soap, composition, or other formulation is available in a solid or semi-solid form (as opposed to a liquid), with the rate of dissolution or degradation of the soap selected for the desired number of uses of the appliance. Soaps, compositions, or formulations in solid or semi-solid form may be attached to the personal-care appliance in some way (for example, solid soaps may be encased in a porous or permeable material such that the solid soap is accessible to water during use of the personal care appliance). In this way, the substrate or personal-care appliance may be adapted for about 1 to about 5 uses; suitably from about 2 to about 7 uses; or for less than about 10 uses.

Any method for applying or associating a composition or formulation with the appliance may be used, so long as the composition or formulation is adapted, at least in part, to be released from the appliance during use thereof by a user of the appliance.

### Representative Packages Comprising a Two-Sided Personal-Care Appliance of the Present Invention

The manufacturer of a two-sided personal-care appliance of the present invention (whether a washing and/or exfoliating and/or moisturizing buff or pad or other such appliance) may fashion messages, statements, or copy to be transmitted to a purchaser, consumer, or user of said appliance. Such messages, statements, or copy may be fashioned to help facilitate or establish an association in the mind of a user of the appliance between an appliance of the present invention, or use thereof, and one or more mental states, psychological states, or states of well being. The communication, statements, or copy may include various alphanumeric strings, including, for example: relax, peace, energy, energize, sex, sensuality, sensual, spa, spirit, spiritual, clean, fresh, mountain, country, zest, sea, sky, health, hygiene, water, waterfall, moisture, moisturize, derivatives or combinations thereof, or other such states. In one embodiment, the communication, statements, or copy create a mental association in the mind of the consumer between a two-sided personal-care appliance of the present invention, and a spa or spa-related experience.

Alphanumeric strings like those referred to above may be used either alone, adjacent to, or in combination with, other alphanumeric strings. The communication, statements, message, or copy could take the form of (i.e., be embodied in a medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers or potential consumers. Other exemplary versions of such communications, statements, messages, and/or copy may be found in, for example, U.S. Pat. Nos. 6,612,846 and 6,896,521, both entitled “Method for Displaying Toilet Training Materials and Display Kiosk Using Same”; co-pending U.S. application Ser. No. 10/831476, entitled “Method of Enunciating a Pre-Recorded Message Related to Toilet Training in Response to a Contact”; co-pending U.S. application Ser. No. 10/956763, entitled “Method of Manufacturing and Method of Marketing Gender-Specific Absorbent Articles Having Liquid-Handling Properties Tailored to Each Gender”; each of which is incorporated by reference in their entirety in a manner consistent herewith.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing appliances of the present invention, the materials of construction of said package may be selected to reduce, impede, or eliminate the passage of water or water vapor through at least a portion of the package. Alternatively, the package may be selected to facilitate transmission of water vapor.

As noted above, some embodiments of the present invention comprise a cleaning composition, moisturizing composition, some combination thereof, and the like. Such compositions may contain water. Therefore packages, containers, envelopes, bags, and the like that reduce, minimize, or eliminate the evaporation or transmission of water or water vapor from appliances contained therein may be beneficial. Furthermore, appliances may be individually wrapped in containers, packets, envelopes, bags, or the like that inhibit, reduce, or eliminate the passage or transmission of water or water vapor from appliances contained therein. For purposes of this application, “packages,” “containers,” “envelopes,” “bags,” “packets,” and the like are interchangeable in the sense that they refer to any material adapted to enclose and hold either individual appliances (as in, for example, an individual packet containing a single appliance), or a plurality of appliances (as in a flexible bag made of film containing a plurality of appliances, whether or not each of the individual appliances are enclosed and held in a separate material—such as individual packets).

In other versions of the invention, materials for constructing packages, containers, envelopes, bags, packets, and the like are selected so that the transmission of water or water vapor is facilitated. This may be the case where systematic drying of a two-sided personal-care appliance comprising a water-based cleaning composition is desired after the appliance's manufacture.

In some embodiments of the present invention, a package will contain not only one or more two-side personal-care appliances of the present invention, but other personal-care products. In one embodiment, a personal-care appliance of the present invention, such as a cleaning and/or exfoliating and/or moisturizing buff or pad, is sold, transferred, distributed, or marketed with other products directed to personal-care, especially products directed to cleaning, moisturizing, or otherwise caring for a user's skin. For example, a two-sided personal-care appliance of the present invention can be sold, transferred, distributed, or marketed with a personal-care appliance for moisturizing a user's skin (e.g., hand, foot, forearm, or other locations on a user's body). A co-pending U.S. Patent Application (U.S. patent application Ser. No. 11/190,597) entitled “Appliance for Delivering a Composition,” filed on 26 Jul. 2005 to K. Close et al., describes such appliances, including socks comprising compositions for moisturizing feet, and gloves comprising compositions for moisturizing hands. This application is hereby incorporated by reference in its entirety in a manner consistent herewith. In another version of the invention, a two-sided personal-care appliance of the present invention is sold with a substrate or personal-care appliance comprising said substrate, e.g., a pouf having an appearance of a naturally-occurring sea sponge. A co-pending U.S. Patent Application (U.S. Patent Application Number not yet assigned; internal docket number K-C 21999) entitled “Substrate And Personal-Care Appliance For Health, Hygiene, And/Or Environmental Application(s); And Method Of Making Said Substrate And Personal-Care Appliance,” filed on 1 Nov. 2005 to K. Close et al., describes such appliances, including a pouf. This application is hereby incorporated by reference in its entirety in a manner consistent herewith. Other combinations of such personal-care appliances are possible and within the scope of the present invention. It should be noted that such combinations may be marketed and packaged as described in the preceding paragraphs. In one version of the invention, these combinations are marketed in such a way that the design, function, and/or appearance of the individual products making up the combination are related to a common theme. One theme, for example, may be that each product provides a spa-like, or spa-related, treatment or experience for the user of the products. “Spa-like” or “spa-related” relates or refers to a fashionable and/or beneficial treatment or experience analogous to a treatment or experience a guest might receive at a resort, hotel, or other such establishment where a person is refreshed, seeks relaxation, seeks beneficial treatments of his or her skin, hair, muscles, finger nails, toe nails, face, or other parts of the body, and the like.

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

### EXAMPLES

### Example 1

Eleven ounce-per-square-yard Spectrum Spunbond, a high-loft, compressible material, was obtained from Kimberly-Clark Corporation. This material was made as described above regarding the second interbonded fibrous layer.

The first interbonded fibrous layer was made as described above without reinforcing strands/filaments. This material was made using a meltblown unit operation as described above. One version of this layer was made with a dry blend of 97% by weight Achieve 3854, a peroxide-free polypropylene (ExxonMobil, 4999 Scenic Highway, Baton Rouge, La.) and 3% by weight Jade pigment (SCC 05SAM06233 is produced by Standridge Color Corporation, 1196 Hightower Trail, Social Circle, Ga. 30025). Another version of this layer was made with a dry blend of: 48.5% by weight Pro-Fax PF-015, a polypropylene material (Basell USA Inc., 4101 Hwy 108 Westlake); 48.5% Achieve 3854 (ExxonMobil); and 3% Jade Pigment (SCC 05SAM06233 is produced by Standridge Color Corporation). A third version of this layer was made with a dry blend of: 87% by weight Pro-Fax PF-015 (Basell USA); 10% Vistamaxx PITD 1816; and 3% Jade Pigment (SCC 05SAM06233 is produced by Standridge Color Corporation).

To make substrates of the present invention, conveyor belts were obtained from Midwest Industrial Rubber, a business having offices at W6470 Levi Drive, Greenville, Wis. For the prepared substrates, the acquired belts were 15.5 inches wide and 75 inches long (with the belt ends joined together to form an endless belt). The procured belts each had a textured surface. The belts were modified by the manufacturer, in accordance with our specifications, to include die-cut circular holes having a diameter of 0.25 inches. The centers of the die-cut holes were 0.375 inches apart in the width dimension of the belt; and the rows were 0.375 inches apart in the length dimension of the belt. The model numbers (with manufacturer's description in brackets) of the acquired belts were MIR 7118 [silicone; endless belt]; MIR 1133 [green RT rough-top; endless belt] (the belt used to make the first interbonded fibrous layer as described below); MIR 1111 [white, negative profile; endless belt]; and MIR 1139 [tan, diamond-top; endless belt].

First interbonded fibrous layers of the present invention were made using a process like that depicted in FIG. 2. The tips of the capillaries from which the interbonded fibrous layer was formed were about 8 inches from the surface of the moving support. Furthermore, the individual capillaries in the meltblowing die were arranged such that there were 30 holes per inch in a direction transverse to the direction of movement of the support (with a total of 12 inches worth of holes in a direction transverse to the direction of movement of the support). These die capillaries had a diameter of about 0.0145 inch.

Polymeric ingredients for the interbonded fibrous layer were added to a hopper coupled to an extruder. These polymeric ingredients were then progressively heated until they were blended and had reached a temperature of about 500 degrees Fahrenheit. Polymeric fibers were then formed by directing the molten polymeric material through the capillaries. For these versions of the first interbonded fibrous layers, the primary air temperature of the air used to form the meltblown material was about 600 degrees Fahrenheit for code 1; 500 degrees Fahrenheit for codes 2 and 3. The pressure at which the primary air flow was directed through the meltblowing die was about 28 pounds per square inch. The below-wire vacuum or exhaust was 7 inches of water for all three codes.

The process parameters corresponding to the making of the first interbonded fibrous layer are given below. (“Mb melt temp” gives the temperature, in degrees Fahrenheit, of the meltblown material at a location proximate to its exiting from the capillaries; “Mb Primary Air Temp” gives the temperature, in degrees Fahrenheit, of the heated air that flows around the meltblown material as the material exits the capillaries; “MB Primary Air Pressure” gives the pressure, in pounds per square inch, of the heated air that flows around the meltblown material as the material exits the capillaries—the location at which this pressure was measured is upstream from the bank of capillaries and closer to the compressor source, and therefore higher than the expected 2-3 pounds per square inch of pressure at a location proximate to the location at which the air actually flows around meltblown material exiting the capillaries; “Mb PIH” refers to the pounds [mass] of meltblown material exiting one linear inch of capillaries, in the transverse direction, per hour; “Line Speed” gives the linear velocity, in feet per minute, of the moving support/belt as it moves in a direction transverse to the banks of capillaries through which the interbonded fibrous layer—here, a meltblown material—is formed; “Filament PIH” refers to the pounds [mass] of any optional filament/reinforcing strand exiting one linear inch of capillaries, in the transverse direction, per hour; “Filament Melt Temp.” gives the temperature of any optional filament/reinforcing strand at a location proximate to the strands' exiting from the corresponding bank of capillaries; “Filament:Mb Ratio” gives the ratio of the Filament PIH to the Mb PIH; “Basis Weight” gives the weight of the resulting substrate in grams per square meter.)

TABLE 1

| | | Mb | Mb | | | | | | |
|---|---|---|---|---|---|---|---|---|---|
| | Mb | Primary | Primary | | | | Filament | | |
| | melt | Air | Air | | Line | | Melt | | Basis |
| | temp | Temp | Pressure | Mb | Speed | Filament | Temp. | Filament:Mb | Weight |
| Code* | (F) | (F) | (PSI) | PIH | (FPM) | PIH | (F) | Ratio | (gsm) |
| 1 | 500 | 600 | 28 | 0.75 | 5 | NA | NA | 0:100 | 150 |
| 2 | 500 | 500 | 28 | 0.75 | 5 | NA | NA | 0:100 | 150 |
| 3 | 500 | 500 | 28 | 0.75 | 5 | NA | NA | 0:100 | 150 |
| 4** | 500 | 500 | 28 | 0.75 | 10 | 0.75 | 425 | 50:50 | 150 |
| *For codes 1-3, a dye was not used. The polymeric composition of each of these first interbonded fibrous layers was: code 1, 100% by weight Achieve 3854; code 2, 50% by weight Achieve 3854 and 50% by weight Pro-Fax PF-015; code 3, 90% by weight Pro-Fax PF-015; 10% Vistamaxx PITD 1816. Codes made with dye were made under analogous process conditions, and had the compositions stated in the first paragraph of this Example 1. “NA” denotes “not applicable,” in | | | | | | | | | |
| # that reinforcing strands were not formed for codes 1, 2, and 3. | | | | | | | | | |
| **Prophetic Example. Polymers to be used in Prophetic Example 4: Meltblown/first interbonded fibrous layer = 97% by weight Achieve 3854 (ExxonMobil, 4999 Scenic Highway, Baton Rouge, LA) and 3% by weight Jade pigment (SCC 05SAM06233 is produced by Standridge Color Corporation. Filament/reinforcing strand = Vistamaxx PLTD 1816 (ExxonMobil; a blend of polyethylene/polypropylene elastomers made with a metallocene catalyst). For additional information on making an interbonded | | | | | | | | | |
| # fibrous layer with filaments/reinforcing strands, see co-pending U.S. Patent Application (U.S. Patent Application Number not yet assigned; internal docket number K-C 21999) entitled “Substrate And Personal-Care Appliance For Health, Hygiene, And/Or Environmental Application(s); And Method Of Making Said Substrate And Personal-Care Appliance” filed on 1 Nov. 2005 to K. Close et al. This application is hereby incorporated by reference in its entirely in a manner consistent herewith. | | | | | | | | | |

The first interbonded fibrous layer was bonded to the second interbonded fibrous layer using SA-15, a polypropylene-based adhesive available from Huntsman Polymer, a business having offices in Houston, Texas. This adhesive is described in the following co-pending United States patent applications and patents, each of which is incorporated by reference in a manner consistent herewith: US20050054780 A1; U.S. Pat. No. 6,774,069 B2; U.S. Pat. No. 6,872,784 B2; and U.S. Pat. No. 6,887,941 B2. Other adhesives, including hot-melt adhesives, may be used, including H2840, an adhesive available from Bostik Findley.

For these representative examples, conventional hot-melt-adhesive processing equipment was used to heat the SA-15 adhesive to a temperature of about 400 degrees Fahrenheit so that it would flow. The molten adhesive was then conducted to a meltblown spray tip to spray the adhesive onto a surface or face of the high-loft Spectrum spunbond material identified above (i.e., the second interbonded fibrous layer). Both the Spectrum spunbond material and the meltblown, first interbonded fibrous layer were in roll form, and were unwound at equal speeds, with the line operating at a speed of 50 feet per minute. The adhesive was applied at an add-on level of 20 grams per square meter. Almost immediately after the adhesive was applied (less than about 1-2 sec), the surface or face of the high-loft Spectrum spunbond material to which the adhesive was applied was joined to a surface or face of the first interbonded fibrous layer, with the combination directed to a nip between two rolls, with the gap between the two rolls being one-half inch. We chose this gap width to apply sufficient pressure to join the two materials together, but without unduly compressing the resulting laminate (especially the high-loft material). The resulting laminate was then wound up.

An egg-shaped die was then used to cut two-sided, personal-care appliances from the laminate. The resulting appliance was adapted for both exfoliating and/or stimulating and/or gently abrading skin; and for gently cleaning skin.

### Example 2

The following ingredients were obtained from the identified supplier, and combined as indicated in the text following the table below.

| | Raw Material | % w/w | Vendor |
|---|---|---|---|
| 1 | Surfactant Blend | 50.00 | Cognis |
| | (55.7% Decyl Glucoside, 17% | | Ambler, PA |
| | Cocamidopropyl Betaine, 20% | | |
| | Glycerin, 5% PEG-7 Glyceryl | | |
| | Cocoate, 0.25% DMDM Hydantoin, | | |
| | 0.25% Iodopropyl Butylcarbamate, | | |
| | 0.45% Citric Acid, 1.35% Water) | | |
| 2 | Plantapon ACG 50 | 10.00 | Cognis |
| 3 | Mackadet CA | 10.00 | McIntyre |
| | | | University Park, |
| | | | IL |
| 4 | Glycerin, 99.5% USP | 6.00 | Glenn Corp. |
| | | | St. Paul, MN |
| 5 | Water, USP | 4.147 | |
| 6 | 1,3, Butylene Glycol | 3.20 | Ruger Chemicals |
| | | | Linden, NJ |
| 7 | Lamesoft PO 65 | 3.00 | Cognis |
| 8 | Polyquart 701 NA | 2.00 | Cognis |
| 9 | Elestab FL-15 | 2.00 | Cognis |
| 10 | Actiphyte of Avocado BG50P | 2.00 | Active Organics |
| | | | Lewisville, TX |
| 11 | Actiphyte of Aloe Vera 10 fold | 2.00 | Active Organics |
| | BG50P | | |
| 12 | Actiphyte of Jojoba Meal BG50P | 2.00 | Active organics |
| 13 | Fragrance | 1.20 | |
| 14 | Tinoderm A | 1.00 | Ciba Specialty |
| | | | High Point, NC |
| 15 | dl-Panthenol, USP | 1.00 | Ruger Chemicals. |
| 16 | Citric Acid | 0.353 | Sigma |
| | | | St. Louis, MO |
| 17 | Vitamin E Acetate, USP | 0.10 | Ruger Chemicals |
| | TOTAL | 100.0 | |

The recited proportions of decyl glucoside, cocamidopropyl betaine, glycerin, PEG-7 glyceryl cocoate, DMDM hydantoin, lodopropyl butylcarbamate, and a solution of the citric acid and water mixed together, in the recited sequence, in a Lightnin Labmaster mixer LIU10F (135 Mt. Read Blvd., Rochester, N.Y.). To this surfactant solution was added and dispersed 98% of the recited mass of water and ingredients 2 through and including 12, 14, and 17. The recited amount of panthenol was then mixed with 1% of the recited mass of water and dissolved. This panthenol mixture was then added to, and mixed with, the mixture prepared earlier. One percent of the recited mass of water was then combined with the identified citric acid ingredient. The resulting citric acid solution was then used to adjust the pH of the completed mixture to between 5.5 and 6.5. Fragrance was then added to, and dispersed in, the completed, pH-adjusted, mixture.

The cleaning composition was then applied to a personal-care appliance of the present invention, in this case by applying 4 grams of the cleaning composition relatively uniformly to the surface of the high-loft substrate (i.e., the second interbonded fibrous layer) of the personal-care appliance (code 1 described in Example 1 above). The personal-care appliance was then placed on a flat surface, with the second interbonded fibrous layer facing up, to allow the cleaning composition to penetrate into the appliance. The resulting personal-care appliance treated with the cleaning composition described above is adapted for the formation of lather useful for cleaning and/or treating and/or moisturizing the skin; and for exfoliating and/or stimulating and/or gently abrading the skin.

### Example 3

The following ingredients were obtained from the identified supplier, and combined as indicated in the text following the table below.

| | Raw Material | % w/w | Vendor |
|---|---|---|---|
| 1 | Surfactant Blend | 50.00 | Cognis |
| | (55.7% Decyl Glucoside, 17% | | |
| | Cocamidopropyl Betaine, 20% | | |
| | Glycerin, 5% PEG-7 Glyceryl | | |
| | Cocoate, 0.25% DMDM Hydantoin, | | |
| | 0.25% Iodopropyl Butylcarbamate, | | |
| | 0.45% Citric Acid, 1.35% Water) | | |
| 2 | Plantapon ACG 50 | 10.00 | Cognis |
| 3 | Mackadet CA | 10.00 | McIntyre |
| 4 | Glycerin, 99.5% USP | 6.00 | Glenn Corp. |
| 5 | Water, USP | 4.147 | |
| 6 | 1,3, Butylene Glycol | 3.20 | Ruger Chemicals |
| 7 | Lamesoft PO 65 | 3.00 | Cognis |
| 8 | Polyquart 701 NA | 2.00 | Cognis |
| 9 | Elestab FL-15 | 2.00 | Cognis |
| 10 | Actiphyte of Avocado BG50P | 2.00 | Active Organics |
| 11 | Actiphyte of Aloe Vera 10 fold | 2.00 | Active Organics |
| | BG50P | | |
| 12 | Actiphyte of Jojoba Meal BG50P | 2.00 | Active organics |
| 13 | Fragrance | 1.20 | |
| 14 | Tinoderm A | 1.00 | Ciba Specialty |
| | | | Chemicals |
| 15 | dl-Panthenol, USP | 1.00 | Ruger Chemicals |
| 16 | Citric Acid | 0.353 | Sigma |
| 17 | Vitamin E Acetate, USP | 0.10 | Ruger Chemicals |
| 18 | Jojoba Spheres 20 | 4.0 | Desert Whale |
| | | | Jojoba Company, |
| | | | Inc., Tuscon, AZ |
| 19 | Microscrub 20 | 2.0 | Presperse Inc., |
| | | | Somerset, NJ |
| | TOTAL | 100.0 | |

The recited proportions of decyl glucoside, cocamidopropyl betaine, glycerin, PEG-7 glyceryl cocoate, DMDM hydantoin, lodopropyl butylcarbamate, and a solution of the citric acid and water mixed together, in the recited sequence, in a Lightnin Labmaster mixer LIU10F (135 Mt. Read Blvd., Rochester, N.Y.). To this surfactant solution was added and dispersed 98% of the recited mass of water and ingredients 2 through and including 12, 14, and 17. The recited amount of panthenol was then mixed with 1% of the recited mass of water and dissolved. This panthenol mixture was then added to, and mixed with, the mixture prepared earlier. One percent of the recited mass of water was then combined with the identified citric acid ingredient. The resulting citric acid solution was then used to adjust the pH of the completed mixture to between 5.5 and 6.5. Fragrance was then added to, and dispersed in, the completed, pH-adjusted, mixture.

The cleaning composition was then applied to a personal-care appliance of the present invention, in this case by applying 4 grams of the cleaning composition relatively uniformly to the surface of the high-loft substrate (i.e., the second interbonded fibrous layer) of the personal-care appliance (code 1 described in Example 1 above). The personal-care appliance was then placed on a flat surface, with the second interbonded. fibrous layer facing up, to allow the cleaning composition to penetrate into the appliance. The resulting personal-care appliance treated with the cleaning composition described above is adapted for the formation of lather useful for cleaning and/or treating and/or moisturizing the skin; and for exfoliating and/or stimulating and/or gently abrading the skin.

### Example 4

### Physical Characterization of Versions of Two-Sided Personal-Care Appliance of the Present Invention

The diameter of a high-loft second interbonded fibrous layer (e.g., the Spectrum material referred to in Example 1) was evaluated by image analysis. The mean diameter of the fiber in this layer was 18 micrometers (with a standard deviation of 1 micrometer).

The diameters of two examples of a first interbonded fibrous layer were determined. These layers were made as generally described in Example 1 and in the specification. One example comprised fiber having a mean diameter of 23 micrometers (with a standard deviation of 2 micrometers). A second example comprised fiber having a mean diameter of 57 micrometers (with a standard deviation of 10 micrometers). The diameter was determined by measuring the distance along a line perpendicular to the outer perimeter (sides) of a fiber. The distance equated to the distance between the two sides in the two-dimensional image.

The size of pores defined by interbonded fiber in each of the interbonded fibrous layers was determined. The equivalent circular diameter was determined for 3 replicate analyses, with each analysis including 300-100 individual measurements. The mean equivalent circular diameter for the pores defined by fiber in the high-loft second interbonded fibrous layer was 75 micrometers (with a standard deviation of 12 micrometers). The mean equivalent circular diameter for the pores defined by fiber in the one example of a first interbonded fibrous layer (made as described in Example 1) was 57 micrometers (with a standard deviation of 3 micrometers). The mean equivalent circular diameter for the pores defined by fiber in a second example of a first interbonded fibrous layer (made as described in Example 1) was 163 micrometers (with a standard deviation of 6 micrometers). Additional detail regarding analyses of equivalent circular diameter is given in U.S. Pat. No. 4,798,603, entitled “Absorbent Article Having a Hydrophobic Transport Layer” and listing Stephen Meyer, et al., as inventors, which is hereby incorporated by reference in its entirety in a manner consistent herewith. For purposes of this application, this measurement corresponds to the term “mean pore diameter” or “mean pore size.”

Generally a first interbonded fibrous layer comprising fiber defining pores having a mean pore diameter greater than the mean pore diameter of the second interbonded fibrous layer is preferred because the larger pores are believed to be better able to receive debris, skin and otherwise, removed from a skin surface during exfoliation. Also, generally the first interbonded fibrous layer comprises fiber having a larger mean diameter than the mean diameter of fiber in the second interbonded fibrous layer. A larger mean diameter generally corresponds to a stiffer fiber better suited to facilitate exfoliation and/or stimulation of skin.

We claim:

1. A two-sided personal-care appliance comprising:
a first interbonded fibrous layer comprising a first face and a second face opposing said first face, each face of said first interbonded fibrous layer having a three-dimensional topography, and wherein the first interbonded fibrous layer defines a first mean pore size and includes fiber having a first mean diameter; and
a second interbonded fibrous layer comprising a first face and a second face opposing said first face, wherein the first face of the second interbonded fibrous layer is attached to and substantially conforms to said three-dimensional topography of said second face of said first interbonded fibrous layer, and wherein the second interbonded fibrous layer defines a second mean pore size and comprises fiber having a second mean diameter.

2. The two-sided personal care appliance of claim 1 wherein the first interbonded layer defines shaped discontinuities.

3. The two-sided personal care appliance of claim 1 wherein the first interbonded layer further comprises reinforcing strands.

4. The two-sided personal care appliance of claim 2 wherein the shaped discontinuities are circular depressions or openings in the first interbonded fibrous layer.

5. The two-sided personal care appliance of claim 1 further comprising an adhesive between the second face of the first interbonded fibrous layer and the first face of the second interbonded fibrous layer.

6. The two-sided personal care appliance of claim 1 further comprising a cleaning composition.

7. The two-sided personal care appliance of claim 6 wherein the cleaning composition is located in the second interbonded fibrous layer.

8. The two-sided personal care appliance of claim 1 wherein the first mean pore size is greater than the second mean pore size and the first mean diameter is greater than the second mean diameter.

9. A method of making a two-sided personal-care appliance, the method comprising the steps of:
(a) forming a first interbonded fibrous layer on a moving support having openings;
(b) moving at least some portion of the first interbonded fibrous layer proximate to at least some of said openings into at least some of said openings thereby forming shaped discontinuities in said interbonded fibrous layer;
(c) attaching said first interbonded fibrous layer to a high-loft nonwoven substrate.

10. The method of claim 9 wherein the moving support has a textured surface, and wherein the textured surface imparts a three-dimensional topography to the first interbonded fibrous layer.

11. The method of claim 9 further comprising the steps of forming reinforcing strands and attaching at least some portion of said strands to at least some portion of the first interbonded fibrous layer.

12. A package, the package comprising:
a container; and
one or more two-sided personal-care appliances of claim 1 contained in said container.

13. The package of claim 12 wherein the container is impermeable to water and water vapor.

14. The package of claim 13 wherein each personal-care appliance is further contained in a separate envelope, and wherein each envelope is impermeable to water and water vapor.

15. The package of claim 12 further comprising a statement on or in the package, said statement relating the appliance to spa-related activities or experiences.

16. The package of claim 12 further comprising a statement on or in the package referring to the appliance being adapted for limited use by a user of the appliance.

17. The package of claim 12 further comprising one or more of the following alphanumeric strings: relax, peace, energy, energize, sex, sensuality, sensual, spa, spirit, spiritual, clean, fresh, mountain, country, zest, sea, sky, health, hygiene, water, waterfall, moisture, moisturize, or some combination thereof.

18. The package of claim 12 further comprising a personal-care appliance for moisturizing skin, a personal-care appliance for cleansing skin, or both.

19. The package of claim 18 wherein the personal-care appliance for cleansing skin is a pouf, and wherein the personal-care appliance for moisturizing skin is a sock for moisturizing the foot or a glove for moisturizing the hand.

20. The package of claim 12 wherein the container is permeable to water and water vapor to facilitate transmission of water or water vapor from personal-care appliances contained therein.

21. The package of claim 12 wherein each personal-care appliance is further contained in a separate envelope, and wherein each envelope is permeable to water and water vapor to facilitate transmission of water and water vapor from personal-care appliances contained therein.
